(19) United States
(12) Patent Application Publication
(10) Pub. No.: US 2025/0228767 A1
(43) Pub. Date: Jul. 17, 2025

(54) COMPOSITIONS AND METHODS FOR DRUG INSTILLATION INTO THE URINARY BLADDER

(71) Applicant: TRIGONE PHARMA LTD., Raanana (IL)

(72) Inventors: Avigdor GORDON, Herzliya (IL); Nadav MALCHI, Raanana (IL); Taher NASSER, TurAan (IL); Dan TOUITOU, Zichron Yaakov (IL)

(21) Appl. No.: 18/701,630

(22) PCT Filed: Oct. 14, 2022
(86) PCT No.: PCT/IL2022/051090

§ 371 Date: Apr. 16, 2024

Related U.S. Application Data
(60) Provisional application No. 63/256,583, filed on Oct. 17, 2021.

(51) Int. Cl. A61K 9/00; A61K 31/337; A61K 31/7068; A61K 45/06; A61K 47/18; A61K 47/32; A61K 47/34; A61K 47/36; A61P 13/10; A61P 35/00

(52) U.S. Cl. CPC A61K 9/0024; A61K 9/0034; A61K 31/337; A61K 31/7068; A61K 45/06; A61K 47/18; A61K 47/32; A61K 47/34; A61K 47/36; A61P 13/10; A61P 35/00

(57) ABSTRACT

The invention relates to a biphasic composition for instillation into a body cavity, such as the urinary bladder, or kidney for delivering an active agent comprising Phase A, wherein Phase A is a hydrophilic hydrogel; and Phase B, is a liquid organic solution of hydrophobic polymer, silicon or a wax (Phase B) comprises wherein a core is formed in-situ in the urinary bladder upon instillation of Phase B into Phase A and wherein the formed core entraps and allows an extended release of the active agent. Further provided are methods of and kits for delivering an active agent into a body cavity and methods for treating or ameliorating diseases or conditions related to the urinary bladder.

### TECHNOLOGICAL FIELD

The invention relates to compositions and methods for instilling drugs and active agents into the body cavities, such as, the urinary bladder.

### BACKGROUND OF THE INVENTION

The urinary bladder is a muscular, hollow pelvic organ whose main functions include the storage and expulsion of urine. The relative impermeability of the bladder epithelium minimizes the systemic absorption of the drug and the side effects. The bladder is easily accessible using a catheter or cystoscope through the urethra by a simple procedure that can be performed by health care practitioners or even by patients themselves. However, drugs or agents instilled intravesical into the bladder have limited efficacy due to periodic dilution and wash-out during urine formation and voiding. This decreases the amount of time a drug is in contact with the targeted tissue. This method of delivery is cumbersome, as it requires repeated and frequent bladder catheterization and drug instillation. Consequently, research has focused on increasing the dwell time and absorption of intravesical drugs with the development of novel intravesical drug therapy systems.

Diseases of the urinary bladder, such as bladder carcinomas and interstitial cystitis, cause acute damage to the bladder wall and cannot be effectively treated by systemic administration of drugs. Such conditions may benefit from intra-bladder drug delivery (IBD), which involves direct instillation of drug into the bladder via a catheter, to attain high local concentrations of the drug for prolong periods with minimal systemic effects. For example, the use of oral oxybutynin for treating overactive bladder is well documented, but despite its clinical efficacy, the systemic anticholinergic side-effects caused by the oral administration of Oxybutynin are marked. One of the key differences distinguishing the delivery methods appears to be in the ratio of parent compound Oxybutynin to the active metabolite N-desethyloxybutynin (NDO) responsible for many of the anticholinergic side effects associated with Oxybutynin. Oxybutynin undergoes extensive upper gastrointestinal first-pass metabolism leading to high NDO concentrations. Local Oxybutynin instillation to the bladder will bypass the first pass effect, reducing the systemic side effects related with NDO.

Current IBD technologies however have their limitations and challenges. The urinary bladder possesses challenges for the design of an efficient IBD due to the high volume of water in the bladder cavity, frequent urination and the contraction and relaxation of the detrusor smith muscle during filling and micturition. Instilled drug solutions are diluted by urine and washed out of the bladder during voiding, necessitating repeated infusions of the drug. Other challenges for IBD technologies include relative short retention time of drugs within the bladder, which require frequent instillation reducing effect and patient compliance, issues regarding catheter or urethra obstruction during instillation, pain, patient tolerability during IBD of readymade matrixes, devices and solids. Other remaining challenges include high volume of the instilled drug formulation.

### SUMMARY OF THE INVENTION

According to some embodiments, there is provided an advantageous biphasic composition for delivering an active agent to an internal cavity, such as the urinary bladder.

This invention relates to a biocompatible and biodegradable delivery system having a sufficient size that serves as a reservoir of one or more active agents released at a sustained rate into a body cavity and more specifically the urinary bladder.

According to some embodiments, a biphasic composition for in-situ drug delivery system formation within the urinary bladder is provided, comprising phase A, which is a hydrophilic hydrogel and phase B, of a liquid comprising an organic solvent, a hydrophobic polymer, a wax, silicone or a lipid and an active agent, wherein Phase A is instilled into the urinary bladder followed by instillation of phase B into Phase A to form, in-situ, a solid entrapping, allowing the sustained release of the one or more active agent. The delivery system releases the active agent(s) over an extended period of time. Phase A facilitates the bulky structure formation of an amorphous spheric mass formed upon instillation of phase B.

According to some embodiments, the novel composition disclosed herein is advantageous, as it is stable, easy to produce, and exhibit a desired biological activity over time, as further detailed herein. In some embodiments, there is provided a composition that enables a controlled release system that may be instilled into a body cavity. In some embodiments, the composition may deliver at least one active ingredient. In some embodiments, the composition may deliver two or more active ingredients. In some embodiments, the instilled composition forms solid bulky scaffolds, three-dimensional (3-D) structures in-situ.

In some embodiments, there is provided a biphasic composition for instillation into the urinary system, urinary bladder, or kidney for delivering an active agent comprising
Phase A, wherein phase A is a hydrophilic hydrogel; and
Phase B comprising an organic solvent and one or more of:
   a hydrophobic polymer;
   a lipid;
   a wax; or
   a silicon
wherein a core is formed in-situ in the internal cavity upon instillation of phase B into Phase A and wherein the formed core entraps and allows an extended release of the one or more active agents.

In some embodiments, Phase A comprising one or more hydrophilic gelling agent selected from the group consisting of carbomer, polyacryl acid, acrylate polymers, carrageenan, polyvinyl alcohol, polyethylene glycol, sodium polyacylatemethylcellulose, hydroxypropyl methylcellulose (HMPC), chitosan, guar gum, xanthan gum, gelatin, water, and optionally an alkali neutralizer.

In some embodiments, the hydrophobic polymer in phase B is polyglycolic acid, polylactic acid, copolymers of polylactic acid (PLA) and polyglycolic acid (PGA), Poly(DL-lactide) poly(lactide-co-glycolide), Poly(L-lactide), Poly(ε-caprolactone) Poly(DL-lactide-co-ε-caprolactone), methacrylic acid-methyl methacrylate copolymer and any combination thereof.

In some embodiments, the organic solvent is DMSO, N-methyl-2-pyrrolidone, ethyl acetate, polyethylene glycol, alcohol, propylene glycol, ethyl oleate, oleic acid, liquid hydrocarbon and any combination thereof.

In some embodiments, the lipid is one or more of a fatty acid, a fatty acid ester, a triglyceride, a glyceride, a phospholipid or a wax.

In some embodiments, the lipid is a wax, bees wax, Witepsol™, lauric acid, cethyl palmitate, and any combination.

In some embodiments, Witepsol™ refers to compounds consist of glycerol esters of vegetable saturated fatty acids, mainly lauric acid. In some embodiments, the Witepsol™ is H, W, S and E.

In some embodiments, the liquid organic solvent of phase B is DMSO. In some embodiments, phase B comprising at least one PLGA co-polymer. In some embodiments, phase B comprising at least two PLGA co-polymers. In some embodiments, the PLGA co-polymers have a monomer ratio composition from 50:50 up to 85:15 poly(lactide-co-glycolide). In some embodiments, the PLGA co-polymers have inherent viscosity range from 0.15 to 1.7 dL/g. In some embodiments, the PLGA co-polymers has acid or hydroxy or ester end groups. In some embodiments, the biphasic composition comprising 0.1-45% w/w PLGA with inherent viscosity range from 0.15-0.25 dL/g; and 0.1-35% w/w PLGA with inherent viscosity range from 0.26 to 0.54 dL/g. In some embodiments, the biphasic composition comprising 0.1-45% w/w PLGA with inherent viscosity range from 0.15-0.25 dL/g; and 0.1-35% w/w PLGA with inherent viscosity range from 0.55 to 0.75 dL/g.

In some embodiments, the biphasic composition comprising an active ingredient, which is an analgesic agent, such as, lidocaine and/or oxybutynin or an anti-cancer agent or any combination thereof.

In some embodiments, the active agent is chosen from the group consisting of mitomycin C, deoxrubicin, valrubicin, cisplatin, gemcitabine, thiotepa, ethoglucid (Epodyl), epirubicin, pirarubicin, apaziquone, docetaxel and vicinium and any combination thereof.

In some embodiments, oxybutynin or any other active ingredient is present in either phase A or phase B at a concentration of 0.02-20% w/w of phase A or phase B. In some embodiments, the biphasic composition comprising the oxybutynin or any other active ingredient is present in either phase A or phase B at a concentration of 0.1-15% w/w of phase A or phase B.

In some embodiments, oxybutynin or solifenacin are present in either phase A or phase B at a concentration of 0.02-5% w/w of phase A or phase B. In some embodiments, lidocaine or bupivacaine are present in either phase A or phase B at a concentration of 0.1-20% w/w of phase A or phase B. In some embodiments, gemcitabine, cisplatin, docetaxel, or paclitaxel are present in either phase A or phase B at a concentration of 0.05-25% w/w of phase A or phase B.

In some embodiments, the biphasic composition comprising active agent that is released continuously for 1, 2, 3, 4, 5, 6, 7, 8, 9, 10, 14, 21, 30 or more days.

In some embodiments, the alkali neutralizer is selected from sodium hydroxide, triethanolamine, diisoprpanoamine, ammonium hydroxide, 2-dimethylamino ethanol, TRIS base, monoisopropanolamine, borax, or any other suitable neutralizer.

In some embodiments, there is provided a method for delivering a therapeutic agent into an internal body cavity of a subject in need thereof, comprising: administering a hydrophilic hydrogel (phase A) into an internal cavity of a subject in need, and administering a liquid organic solution of an active agent and a hydrophobic polymer, a lipid, wax or a silicon (phase B) into the hydrophilic hydrogel (phase A) wherein a core is formed in-situ in the internal cavity upon instillation of phase B into Phase A and wherein the formed solid entraps and allows an extended release of the active agent, wherein phase A or phase B or both comprise at least one active agent; thereby delivering the at least one active ingredient into an internal body cavity. In some embodiments, the core is a solid or semi solid 3D structured formed in phase A and comprises the one or more of a hydrophobic polymer, a silicon, a wax or a lipid. In some embodiments, the active agent is added to phase B during the preparation thereof. In some embodiments, the active agent is added prior to the instillation of phase B into phase A.

In some embodiments, the lipid is one or more of a fatty acid, a fatty acid ester, a triglyceride, a glyceride, a phospholipid or a wax.

In some embodiments, there is provided a method of forming a biphasic composition comprising: dispersing hydrophilic gelling agent in water or any other suitable hydrophilic solvent (phase A); mixing a hydrophobic polymer, a lipid, wax or a silicon or any combination thereof with an organic solvent, until a viscous liquid mixture is obtained (phase B); and injecting phase B into phase A. As mentioned, the active agent may be added to either phase A or phase B or to both.

In some embodiments, there is provided a kit comprising: a hydrophilic hydrogel; a liquid organic solution of hydrophobic polymer, a fatty acid, a fatty acid ester, a silicon, a triglyceride, a glyceride, a phospholipid or a wax; and a leaflet explaining the preparation of in-situ biphasic composition.

In some embodiments, the kit further comprising an active agent.

In some embodiments, there is provided a biphasic composition for drug delivery into a urinary bladder comprising a hydrogel (phase A) and 0.1-45% w/w PLGA with inherent viscosity range from 0.15-0.25 dL/g; 0.1-35% w/w PLGA with inherent viscosity range from 0.26 to 0.54 dL/g or 0.55-0.75 dL/g; 0.1-50% DMSO and an active agent. In some embodiments, the composition further comprising 0.01-20% povidon; 0.01%-0.5% HPMC.

In some embodiments, the active agent is lidocaine, oxybutynin, an anti-cancer agent or any combination thereof. In some embodiments, the active agent is chosen from the group consisting of mitomycin C, deoxrubicin, valrubicin, cisplatin, gemcitabine, thiotepa, ethoglucid (Epodyl), epirubicin, pirarubicin, apaziquone, docetaxel and vicinium. In some embodiments, oxybutynin is present at a concentration of 0.02-5% w/w. In some embodiments, oxybutynin is present at a concentration of 0.01-2% w/w.

In some embodiments there is provided a method of preventing/treating/ameliorating urinary bladder, urinary tract or kidney disease or syndrome comprising the step of administering a hydrophilic hydrogel (phase A) into an the urinary bladder, urinary tract or kidney of a subject in need, and administering an organic solution of active agent and one or more of hydrophobic polymer, a fatty acid, a silicon, a fatty acid ester, a triglyceride, a glyceride, a phospholipid, a silicon or a wax (phase B) into the hydrophilic hydrogel (phase A) wherein a core is formed in-situ in the urinary bladder, urinary tract or kidney upon instillation of phase B into Phase A and wherein the formed amorphous spheric mass entraps and allows an extended release of the active agent, wherein phase A or phase B or both comprises at least one active agent; thereby delivering the at least one active ingredient into the urinary bladder, urinary tract or kidney and treating the urinary bladder, urinary tract or kidney disease or syndrome. In some embodiments, the urinary bladder, urinary tract or kidney disease or syndrome include one or more of urinary tract infection, chronic cystitis, overactive bladder, partial bladder obstruction, interstitial cystitis urethritis, pain and bladder cancer or any of the other syndromes or diseases described herein. The active agent is any of the active agents suggested herein and the physician may choose the appropriate active agent according to the disease or the syndrome.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows the in-vitro continuous release of oxybutynin over 8 days from the in-situ mass generated from the biphasic composition of example 2 into Artificial Urine Fluid (AUF) (Mean+/−SD, n=6).

FIG. 2 shows the in-vitro continuous release of lidocaine over 8 days from the in-situ mass generated from the biphasic composition of example 2 into Artificial Urine Fluid (AUF) (Mean+/−SD, n=6).

FIG. 3 shows the remaining amorphous spheric mass within the bladder 7 days post instillation of composition of example 2 into pig's bladder. Visualisation using a cystoscope.

FIG. 4 shows the pig's mean lidocaine plasma concentration for 8 days following bladder instillation of composition from example 2 (Mean+/−SD, n=4).

FIG. 5 shows the mean oxybutynin and it's metabolite N-desethyloxybutyn in pig's plasma concentration for 8 days following bladder instillation of composition from example 2 (Mean+/−SD, n=4).

FIG. 6 shows the mean oxybutynin in pig's plasma concentration for 10 days following bladder instillation of composition from example 13 (Mean+/−SD, n=2).

FIG. 7 demonstrates the cytotoxic effect of the drugs released from the in-situ mass generated from the composition of example 16 containing docetaxel 0.5% and gemcitabine 0.5% on human urinary bladder cancer T24 cell line in MTT assay.

FIG. 8 demonstrates the cytotoxic effect of the drugs released from the in-situ mass generated from composition of example 17 containing docetaxel 0.1% and gemcitabine 0.1% on human urinary bladder cancer T24 cell line in MTT assay.

FIG. 9 demonstrates the cytotoxic effect of the drugs released from the in-situ mass generated from composition of example 18 containing docetaxel 0.5%, gemcitabine 0.5% and cisplatin 0.5% on human urinary bladder cancer T24 cell line in MTT assay.

FIG. 10 demonstrates the in-vitro continuous release of bupivacaine over 14 days from the in-situ mass generated from the biphasic composition of example 24 into Artificial Urine Fluid (AUF) (Mean+/−SD, n=2).

### DESCRIPTION OF THE DETAILED EMBODIMENTS

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

### Definitions

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

In the description and claims of the application, the words “include” and “have”, and forms thereof, are not limited to members in a list with which the words may be associated.

As used herein, the term “comprising” includes the term “consisting of”.

As used herein, the term “about” may be used to specify a value of a quantity or parameter (e.g. the length of an element) to within a continuous range of values in the neighbourhood of (and including) a given (stated) value. According to some embodiments, “about” may specify the value of a parameter to be between 80% and 120% of the given value. According to some embodiments, “about” may specify the value of a parameter to be between 90% and 110% of the given value. According to some embodiments, “about” may specify the value of a parameter to be between 95% and 105% of the given value.

According to some embodiments, there is provided a biphasic composition for instillation into the urinary system, urinary bladder, urinary tract or kidney, comprising an hydrogel and a solution of one or more polymers, silicone, wax or lipid in an organic solvent and at least one active agent, which composition is forming “in-situ” a core form entrapping the active agent that releases the active agent over an extended period.

In some embodiments, there is provided a biphasic composition for instillation into the urinary system, urinary bladder, or kidney for delivering an active agent comprising
Phase A, wherein phase A comprises a hydrophilic hydrogel; and
Phase B comprising an organic solvent and one or more of: a hydrophobic polymer;
a lipid; a wax; or a silicon; wherein the hydrophobic polymer, lipid, wax or silicon is dissolved in the organic solvent, wherein a core is formed in-situ in the internal cavity upon instillation of phase B into Phase A and wherein the formed core entraps and allows an extended release of the one or more active agents.

In some embodiments, the active agent is added to either phase A or phase B or both prior to the instillation.

According to some embodiments, the organic solvent is DMSO, N-methyl-2-pyrrolidone, ethyl acetate, triacetin, polyethylene glycol, alcohol, propylene glycol, ethyl oleate, oleic acid, liquid hydrocarbon and any combination thereof.

According to some embodiments and the composition further comprises hydroxypropyl methylcellulose and/or polyvinylpyrrolidone.

According to some embodiments, there is provided a biphasic composition for delivering an active agent to an internal cavity, such as the urinary bladder.

According to some embodiments, a biphasic composition for in-situ drug delivery system formation within the urinary bladder is formed, wherein the biphasic composition comprising phase A comprising of a hydrophilic hydrogel and phase B comprising of an organic solvent and a hydrophobic polymer and an active agent, where Phase A is instilled into the urinary bladder, urinary tract or kidney followed by instillation of phase B into Phase A so that in-situ core is formed entrapping the active agent and allowing the extended release of the active agent. In some embodiments, Phase A facilitates the 3-D structure formation of an amorphous spherical mass core formed upon instillation of phase B into phase A.

According to some embodiments, the composition disclosed herein is advantageous, as it is stable, easy to produce, and exhibit a desired biological activity over time, as is further detailed herein. In some embodiments, the mass formed does not obstruct the urethra, i.e., it does not interfere with voiding and elimination of urine.

The administrated dose of active agents can be adjusted by adjusting the administration volume of the composition. The methods described herein also allow for administrating of different active agents by consecutive instillation through a catheter, a cystoscope, or an ureteroscope of compositions containing different active agents. The methods allow for the sequential or simultaneous release of different drugs into the urinary tract, bladder, and/or kidney(s).

According to some embodiments, there is provided a biphasic composition for delivering an active agent to an internal cavity, such as the urinary bladder, urinary tract or kidney wherein the biphasic composition comprising phase A that comprises a hydrophilic colloidal gel that facilitates the in situ 3-D amorphous spherical structure formation upon instillation of Phase B into phase A.

According to some embodiments, phase B includes a solvent containing an organic solvent, such as DMSO, or polyethylene glycol, one or more polymers, and one or more active agents. Optionally, the composition also contains one or more additives. The core formed releases the active agent and provides prolonged release of the active agent into the urinary tract, the bladder, and/or the kidney(s). Release of the one or more active agents, into the urine of the urinary tract, the bladder, or the kidney(s) or any other body cavity, from the core formed can start immediately upon instillation and may continue for few hours up to several weeks. The delivery period in the urinary tract, the bladder, or the kidney(s) can be modulated by the formulation composition (i.e., polymer, solvents, active agents, additives, etc.), the volume instilled, and the frequency of repeated instillations.

The composition may further include at least one pharmaceutically acceptable excipients.

In some embodiments, phase A is a hydrogel comprising one or more hydrophilic gelling agents selected from carbomer, polyacryl acid, acrylate polymer, carrageenan, polyvinyl alcohol, polyethylene glycol, sodium polyacylatemethylcellulose, hydroxypropyl methylcellulose, chitosan, guar gum, xanthan gum, gelatin, alginic acid, hyaluronic acid, water, optionally an alkali neutralizer and any combination thereof.

According to some embodiments, the alkali neutralizer is selected from sodium hydroxide, triethanolamine, diisoprpanoamine, ammonium hydroxide, 2-dimethylamino ethanol, TRIS base, monoisopropanolamine, borax, or any other suitable neutralizer. According to some embodiments, the alkali neutralizer is an active agent.

According to some embodiments, phase A comprises a colloidal hydrogel. In some embodiments, phase A may comprise a one or more pharmaceutically additives. In some embodiments, phase A may contain one or more active agents.

According to some embodiments phase B is a liquid comprising a hydrophobic polymer that can be dissolved in an organic solvent. In some embodiments, the polymer is polyglycolic acid, polylactic acid, copolymers of polylactic acid (PLA) and polyglycolic acid (PGA), Poly(DL-lactide) poly(lactide-co-glycolide), Poly(L-lactide), Poly(ε-caprolactone) Poly(DL-lactide-co-ε-caprolactone), methacrylic acid-methyl methacrylate copolymer and any combination thereof.

According to some embodiments phase B is a liquid comprising lipids, fatty acids, a silicon, a fatty acid ester or waxes that can be dissolved in an organic solvent. In some embodiments, the lipids, fatty acids, fatty acid ester or waxes are bees wax, hard fats, saturated fatty acid triglycerides, Witepsol™, lauric acid, cethyl palmitate, and any combination thereof.

In some embodiments, the organic solvent in phase B is DMSO, N-methyl-2-pyrrolidone (NMP), ethyl acetate, polyethylene glycol, alcohol, propylene glycol, triacetin, ethyl benzoate, triethyl citrate and any combination thereof.

In some embodiments, the organic solvent in phase B is ethyl oleate, oleic acid, liquid hydrocarbon short a, median, triglyceride oils, olive oil, sesame oil, medium-chain triglycerides, triacetin and any combination thereof.

Optionally, the composition contains one or more additives.

Optionally, the composition contains one or more drugs or active substances. Optionally, the composition contains an antibody as an active agent. Optionally, the composition contains a biological molecule. In some embodiments, the active agent is added prior to the instillation.

In some embodiments, there is provided a biphasic composition for instillation into the urinary system, urinary bladder or kidney for delivering one or more active agents comprising of a hydrophilic hydrogel (phase A) and a liquid comprising an organic solution of one or more of a hydrophobic polymer, a lipid, a wax or a silicon, and an active agent (phase B), wherein a core is formed in-situ in the internal cavity upon instillation of phase B into Phase A and wherein the formed core entraps and allows an extended release of the active agent.

In some embodiments, the lipid is one or more of a fatty acid, a fatty acid ester, a triglyceride, a glyceride, a phospholipid or a wax.

In some embodiments, Phase A comprises one or more hydrophilic gelling agent selected from the group consisting of carbomer, polyacryl acid, acrylate polymers, carrageenan, polyvinyl alcohol, polyethylene glycol, sodium polyacylatemethylcellulose, hydroxypropyl methylcellulose, chitosan, guar gum, xanthan gum, gelatin, alginic acid, hyaluronic acid water, and optionally an alkali neutralizer. In some embodiments, phase B comprises hydrophobic polymer which is for example, without being limited polyglycolic acid, polylactic acid, copolymers of polylactic acid (PLA) and polyglycolic acid (PGA), poly(DL-lactide) poly(lactide-co-glycolide), poly(L-lactide), poly(ε-caprolactone), poly(DL-lactide-co-ε-caprolactone), methacrylic acid-methyl methacrylate copolymer and any combination thereof.

In some embodiments, the organic solvent of phase B is DMSO and phase B further comprises at least two PLGA co-polymers.

In some embodiments, the PLGA co-polymers have inherent viscosity range from 0.15 to 0.95 dL/g. In some embodiments, phase B comprises 0.1-45% w/w PLGA with inherent viscosity range from 0.15-0.25 dL/g; and 0.1-35% w/w and PLGA with inherent viscosity range from 0.26 to 0.54 dL/g, from 0.55 to 0.75 dL/g, or from 0.76-1.3 dl/g

In some embodiments, the PLGA co-polymers have acid or hydroxy or ester end groups. In some embodiments, the PLGA co-polymers may have a monomer molar ratio from 50:50 up to 85:15 lactic acid to glycolic acid.

In some embodiments, phase B may contain polylactic acid polymers (PLA), polylactic polyglycolic acid copolymers (PLGA co-polymers), with different molar ratio of the monomers e.g.: 50:50%, 75:25%, 60:40%, 65:35%, 85:15 lactic acid to glycolic acid.

In the embodiments of the invention, the composition may include any pharmaceutically excipient. Illustrative examples include plasticizers, viscosity modifiers, surfactants, enhancing permeation agents, diluents, preservatives, antioxidants, ingredients to facilitate handling, stability, wettability, release kinetics, ingredients needed in the manufacture process or during administration. In some embodiments, active agent or agents in any one of phase A or B can be lipophilic.

In some embodiments, the composition may include any pharmaceutical excipients such as Tween 20, Tween 60, Span 20, Span 80, Chremophore EL, Chremophore RH 40 or Pluronic P85.

In some embodiments, active agent or agents in any one of phase A or B can be hydrophilic or amphiphilic.

In some embodiments, the active agent or agents is dissolved or suspended in any one of phase A or B.

In Some embodiments the active agent can be in the form of liquid, powder, crystals, nanoparticles microspheres or granulates microparticles.

In some embodiments, phase A and or phase B may contain an active agent at a concentration of between: 0.005-30% w/w, 0.01-20% w/w, 0.01-10%, 0.01-5%, 5-10%, 5-15%, 5-20% or 0.1-10% w/w of weight of either phase A or phase B.

In some embodiments, the viscosity of the hydrophilic hydrogel of Phase A at 25° C. is between 200 to 30,000 cP. In some embodiments, the viscosity of the hydrophilic hydrogel of Phase A at 25° C. is between 500 to 20,000 cP. In some embodiments, the viscosity of the hydrophilic hydrogel of Phase A at 25° C. is between 800 to 15,000 cP. In some embodiments, the viscosity of the hydrophilic hydrogel of Phase A at 25° C. is between 800 to 10,000 cP

In some embodiments, the size of the amorphic spheric 3-D mass structure can be modulated by the instilled volume of Phase B, by the polymer used, and the additive concentration.

In some embodiments, phase B of the composition may contain polylactic acid polyglycolic acid copolymers at concentrations of 0.5-60% w/w, 2-45% w/w or 5-35% w/w.

In some embodiments, the composition of phase B may contain polylactic polyglycolic acid copolymers with different molecular weight range.

In some embodiments, the composition of phase B may contain polylactic polyglycolic acid copolymers with acid, hydroxy or ester end groups.

In some embodiments, the composition of phase B may contain a mixture of polylactic polyglycolic acid copolymers with different inherent viscosity.

In some embodiments, the composition of phase B may contain a mixture of 50:50% polylactic polyglycolic acid copolymers with inherent viscosity of 0.15-0.25 dL/g and 0.26-0.54 dL/g. In some embodiments, the composition of phase B may contain a mixture of 50:50% polylactic polyglycolic acid copolymers with inherent viscosity of 0.15-0.25 dL/g and 0.55-0.65 dL/g.

In some embodiments, the composition may contain methyl cellulose at concentrations: 0.01-10% w/w, 0.05-8% w/w or 0.2-7% w/w.

In some embodiments, phase B of the composition may contain a glycol at concentrations: 0-90% w/w, 0-70% w/w or 0.1-60% w/w.

In some embodiments, the composition may contain an amphiphilic polymer at concentrations: 0.1-7% w/w, 0.2-5% w/w or 0.1-4% w/w.

In some embodiments, the in-situ 3-D amorphic spherical core formed has a diameter in the range of between 2 mm-15 cm, between 8 mm-8 cm or between 1-5 cm.

In some embodiments, there is provided a biphasic composition for drug delivery into a urinary bladder comprising a hydrogel and 0.1-45% w/w PLGA with inherent viscosity range from 0.15-0.25 dL/g, 0.1-35% w/w PLGA with inherent viscosity range from 0.26 to 0.54 dL/g or 0.55-0.75 dL/g or 0.76-0.94 dL/g or 0.94-0.1.2 dL/g; and an active agent.

The composition may in some embodiments, further comprise one or more of 0.01-20% povidon; 0.01%-5.0% HPMC; and 0.1-50% DMSO and any combination thereof.

In some embodiments, the composition can be instilled consecutively a number of times to generate multiple bodies with various sizes in the urinary bladder, urinary tract or the kidney.

In some embodiments, the active agent may be released over a period of at least to 1 hour, 2 hours, 5 hours, 8 hours, 24 hours, 48 hours, 7 days, 14 days, 21 days, 30 days or more.

In some embodiments, the active agent is released continuously for 1, 2, 3, 4, 5, 6, 7, 8, 9, 10, 14, 21, 30 or more days.

In some embodiments, the active agent is released continuously for one week, two weeks, three weeks, four weeks or more.

The instillation can be carried out by mean of a catheter, an endoscope or any other suitable means or devices.

In one embodiment the invention relates to a method of administrating the compositions locally to the urinary track, bladder or kidney by instillation, using a catheter, an endoscope or other suitable means or devices.

In one embodiment, the phase A and/or phase B of the composition may incorporate active agents chosen from various therapeutic classes: antinociceptic, antiarrhythmics, anticoagulants, anesthetic, anti-inflammatory, antibiotic, muscarinic, mitotic, chemotherapic; probes for diagnosis, pH buffering agents, radioactive isotopes. Non-limiting example of the active substances include antibiotics, antibodies, cannabinoids, anti-spastics, analgesics, anti-bacterials, antimicotic, antifungals, antihistamines, anti-inflammatories, antineoplastics, antivirals, corticosteroids, cytotoxics, decongestants, diuretics, hormones, immunosuppressives, muscle relaxants, sex hormones, tranquilizers.

In some embodiments, the composition may comprise a combination of drugs. Some none limiting illustrative examples include combinations of cytotoxic drugs e.g. mitomycin and cisplatin, gemcitabine and cisplatin, local anesthesia and antimuscarinic e.g. lidocaine and oxybutynin, bupivacaine and solifenacin, antinociceptic and antiinflammatory drugs, e.g. lidocaine and cannabidiol, phentanyl and cannabidiol, ibuprofen and cannabidiol, THC and Cannabidiol, antimitotic and anesthetic drugs, prilocaine and mitomycin, lidocaine and adriamycin.

In some embodiments, the active ingredient is lidocaine, oxybutynin, an anti-cancer agent or any combination thereof.

In some embodiments, the active agent is chosen from the group consisting of mitomycin C, deoxrubicin, valrubicin, cisplatin, gemcitabine, thiotepa, ethoglucid (Epodyl), epirubicin, pirarubicin, apaziquone, docetaxel, and vicinium and any combination thereof.

In some embodiments, oxybutynin is present in either phase A or phase B at a concentration of 0.02-2% w/w of phase A or phase B, respectively.

In some embodiments, oxybutynin is present in either phase A or phase B at a concentration of 0.1-0.8% w/w of phase A or phase B, respectively.

In some embodiments, oxybutynin or solifenacin are present in either phase A or phase B at a concentration of 0.02-5% w/w of phase A or phase B. In some embodiments, lidocaine or bupivacaine are present in either phase A or phase B at a concentration of 0.1-20% w/w of phase A or phase B. In some embodiments, gemcitabine, cisplatin, docetaxel, or paclitaxel are present in either phase A or phase B at a concentration of 0.05-25% w/w of phase A or phase B.

In some embodiments, there is provided a method for delivering a therapeutic agent into an internal body cavity of a subject in need thereof, comprising: administering a biphasic composition composed of a hydrophilic hydrogel (phase A) into an internal cavity of a subject in need, and administering a liquid organic solution of hydrophobic polymer, silicon or a wax (phase B) (phase B) into the hydrophilic hydrogel (phase A), wherein a core is formed in-situ in the internal cavity upon instillation of phase B into Phase A and wherein the formed solid entraps and allows an extended release of the active agent, wherein phase A or phase B or both comprise at least one active agent; thereby delivering the at least one active ingredient into an internal body cavity.

In some embodiments, the compositions and methods described herein are used for treating diseases and/or conditions in urinary bladder, urinary tract and/or kidney such as, low grade bladder cancer, high grade bladder cancer, non-muscle invasive bladder cancer, muscle invasive bladder cancer, upper tract urothelial cancer, wall superficial cancer; over active bladder, renal colic, bladder interstitial pain, interstitial cystitis, carcinoma, sphincter mal function, infections, incontinence, trigonitis, inflammation, chronic pain, neurogenic bladder, urinary infections, erectile dysfunction,

In some embodiments, the active agent is used for local anesthesia, or is combined with a radioactive agent and may be used for diagnostic purposes.

The active agent may be a therapeutic, prophylactic, or diagnostic agent for treatment of bladder or urinary disorders. In some embodiments, the active ingredient is a small molecule. In some embodiments the active agent is a peptide or a protein or an antibody or a nucleic acid, a virus or a bacteria. The active agent may be a small size molecule drug or a biologic agent, a metabolite or a radioactive molecule. The active agent may be in its salt forms, hydrates, free acid forms and free base forms. The active agent may be solubilized or suspended.

In a preferred embodiment, the active agent is suitable to be administered for the treatment of genitourinary tract, bladder, kidney and prostate ailments.

In yet another embodiment, the compositions and methods described herein are for treating inflammatory conditions such as interstitial cystitis, radiation cystitis, painful bladder syndrome, prostatitis, urethritis, post-surgical pain, and kidney stones. Non-limiting examples of specific drugs for these conditions include lidocaine, bupivacaine, ropivacaine, mepivacaine, levobupivacaine, non-steroidal anti-inflammatory drugs (NSAID), glycosaminoglycans (e.g., chondroitin sulfate, sulodexide), pentosan polysulfate sodium (PPS), dimethyl sulfoxide (DMSO), oxybutynin, mitomycin C, heparin, flavoxate, ketorolac, or a combination thereof. For kidney stones, the drug(s) may be selected to treat pain and/or to promote dissolution of renal stones.

In yet another embodiment, the compositions and methods described herein may treat overactive bladder, bladder incontinence and motility. Example of the active substances for relieving symptoms of overactive bladder and reducing episodes of urge incontinence including: antimuscarinic compounds, antispasmodic agents anticholinergic agents, beta-2 agonists, alpha adrenergics, anticonvulsants, norepinephrine uptake inhibitors, serotonin uptake inhibitors, calcium channel blockers, potassium channel openers, and muscle relaxants, apomorphine, darifenacin, tolterodine, oxybutynin, propiverine, trospium, solifenacin, mirabegron, fesoterodine and analogs thereof alone or in combination with an anesthetic agent.

In yet another embodiment, the compositions and methods described herein may treat bladder or kidney cancer, urothelial carcinoma, squamous cell carcinoma, noninvasive papillary carcinoma, adenocarcinoma, squamous cell carcinoma. Non-limiting example of the active substances or their combination for treating bladder or kidney cancer including: antiproliferative agents, cytotoxic agents, chemotherapeutic agents, immunomodulatory, biologic agents, monoclonal antibody, anti-PD1 antibodies, anti-PD-L1 antibodies, anti 4CB-1 antibodies, anti 4CB-2 antibodies, antibody toxin conjugates, virus, bacteria, a TNF inhibitor, an anti-leukin, kinase inhibitor or a combination thereof, apaziquone, atezolizumab, atezolizumab, avelumab, bavencio, cetrelimab, cisplatin, doxorubicin, durvalumab, enfortumab vedotin, epirubicin, erdafitinib, 5-FU (5-fluorouracil), gemcitabine, human alpha-lactalbumin made lethal to tumor cells” (HAMLET), imfinzi, keytruda, methotrexate, mitomycin C, nivolumab, oportuzumab monatox-qqrs, opdivo, pembrolizumab, pirarubicin, paclitaxel, inactivated Streptococcus pyogenes, tecentriq, thiotepa, tremelimumab, valrubicin, valstar, IL-15 superagonist, IL-15 mutant (IL-15N72D) bound to IL-15 receptor alpha/immunoglobulin G1 (IgG1) crystallizable fragment (Fc) fusion protein, and analogs thereof.

In yet another embodiment, the compositions and methods described herein treat bladder or kidney pain, neurogenic bladder or interstitial cystitis. Non-limiting example of the active substances or their combination for treating pain, bladder pain syndrome or interstitial cystitis including include anesthetic agents, analgesic agents, and combinations thereof, aminoamides, lidocaine base or lidocaine salt, procaine, articaine, benzocaine, bupivacaine, tramadol or dramadol salt, dibucaine, lontocaine, mepivacaine, prilocaine, ropivacaine, tanezumab, gabapentin, chloroprocaine, cocaine, cocaine analogue, proparacaine, tetracaine, cannabinoids, CBD, THC and their combinations, NSAIDs, diclofenac, ibuprofen, naproxen, piroxicam, acetaminophen, flufenisal, indoprofen, indomethacin and analogs thereof. Non limiting examples of opioid agonists include benzylmorphine, buprenorphine, butorphanol, desomorphine, dextromoramide, dezocine, diampromide, diamorphone, dihydrocodeine, dihydromorphine, ethylmorphine, fentanyl, heroin, hydrocodone, hydromorphone, hydroxypethidine, isomethadone, methadone, morphine, myrophine, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tilidine, tramadol, pharmaceutically acceptable salts thereof, and mixtures thereof.

In yet another embodiment, the compositions and methods described herein may treat bladder or kidney inflammation. Non-limiting examples of the anti-inflammatory active substances or their combination for treating bladder urothelium inflammation and pain including cannabinoids, NSAID, diclofenac, Ibuprofen, corticosteroids and analogs thereof.

In yet another embodiment, the composition and methods described herein may treat bladder or kidney infection. Non-limiting examples of the antibiotic active substances or their combination for treating bladder or urinary tract infections including, antibiotics, amoxicillin, ceftriaxone, cephalexin, ciprofloxacin, fosfomycin, levofloxacin, minocycline, nitrofurantoin, trimethoprim/sulfamethoxazole and analogs thereof.

In yet another embodiment, the composition and methods described herein may be used for treating fibrosis of the bladder. Representative examples of drugs for the treatment of fibrosis of the bladder include pentoxphylline, antiTNF, antiTGF agents, GnRH analogues, exogenous progestins, antiprogestins, selective estrogen receptor modulators, danazol and NSAIDs.

In some embodiments of the invention, there is provided a method of forming a biphasic composition according to the embodiments of the invention comprising: dispersing hydrophilic gelling agent in water or any other suitable hydrophilic solvent; mixing liquid organic solution of hydrophobic polymer or a wax until a viscous liquid mixture is obtained (phase B); and injecting phase B into phase A.

In some embodiments, there is provided a kit comprising: a hydrophilic hydrogel; a liquid organic solution of hydrophobic polymer or a wax; and a leaflet explaining the instructions for preparing and instilling the biphasic compositions of the kit. In some embodiments, there is provided a kit comprising: a hydrophilic hydrogel; an organic solution, a hydrophobic polymer, silicon or a wax; and a leaflet explaining the instructions for preparing and instilling the biphasic compositions of the kit. In some embodiments, there is provided a kit comprising: ingredients for phase A; an organic solution and a hydrophobic polymer, a silicon or a wax or any other ingredient for preparing phase B; and a leaflet explaining the instructions for preparing and instilling the biphasic compositions of the kit.

In some embodiments, the kit further comprises an active agent. In some embodiments, the kit further comprises an active agent which is mixed together with phase A, B or both.

In some embodiments, the invention relates to methods of preventing/treating/amilorating urinary tract infection, chronic cystitis, overactive bladder, partial bladder obstruction, interstitial cystitis urethritis, pain and bladder cancer. The invention further relates to reducing the pain and improving the symptoms of patients suffering from pain associated with Endourology Procedures (EUP) or any other procedure.

Interstitial cystitis is a disease that is associated with non-specific chronic inflammation of the bladder and presents symptoms such as frequent urination, increased desire to urinate, urinary urgency, and/or bladder pain, and it produces marked deterioration of quality of life. Interstitial cystitis associated with pain or a condition where interstitial cystitis is suspected is sometimes included among painful bladder syndrome, bladder pain syndrome, or chronic pelvic pain syndrome. The three conditions of (1) the presence of lower urinary tract symptoms such as frequent urination, hypersensitive bladder, and/or bladder pain, (2) the ability to confirm endoscopically lesions of the bladder resulting from Hunner's ulcers and/or bleeding after bladder dilatation, and (3) the fact that other disorders such as infection, malignant tumor, or urinary calculus are excludable is one example of a diagnostic standard for interstitial cystitis.

Overactive bladder (OAB) is a syndrome characterized by symptoms of urgency with or without urge incontinence, usually with frequency and nocturia. OAB is one of several bladder diseases and may also be characterized by urge incontinence, detrusor instability, detrusor hyperreflexia, irritable bladder, spasmodic bladder, unstable bladder, incontinence-urge, or bladder spasms. OAB often presents itself as a strong, sudden need to urinate due to bladder spasms or contractions that can lead to frequent urination, in the daytime and at night, loss of urine (leaking) without meaning to urinate, and the sudden and urgent need to urinate (urinary urgency). Proper bladder control, therefore, requires the lower urinary tract and nervous system to work together to allow for the feeling and ability to respond to the urge to urinate at appropriate intervals, while minimizing leaking and urinary urgency.

The invention relates to methods of treating urinary tract infection, chronic cystitis, overactive bladder, partial bladder obstruction, urethritis and bladder cancer.

In some embodiments, the biphasic composition and the method of delivering an active agent or more of the invention is used for treating urinary tract infection, chronic cystitis, overactive bladder, partial bladder obstruction, urethritis and/or bladder cancer or any of the conditions, symptoms or diseases described herein. As can be seen in the examples, patients suffering from OAB or ICS were

The terms “treating,” “treatment,” or “therapy,” as used herein refer to partially or completely alleviating, ameliorating, improving, relieving, delaying onset of, inhibiting progression of, reducing severity of, reducing incidence of, attenuating one or more of the bladder diseases or symptoms, such as urinary tract infection, chronic cystitis, overactive bladder, partial bladder obstruction, interstitial cystitis urethritis, pain and bladder cancer. Treatment can also induce remission or cure of a condition or can reduce the pathological condition, such as decreasing void volume, decreasing frequency of urination, increasing smooth muscle contractility, reducing bladder ischemia, increasing urethra or bladder contractility, or a combination thereof. Prevention of a disease does not require a total absence of disease.

The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, reduced frequency of urination, increased void volume, a slower progression of the disease, an improvement in the overall health or well-being of the subject, or by other parameters well known in the art that are specific to the particular disease.

The term “treat”, “treating”, or any variation thereof is meant to include therapy utilized to remedy a health problem or condition in a patient or subject. In one embodiment, the health problem or condition may be eliminated permanently or for a short period of time. In another embodiment, the severity of the health problem or condition, or of one or more symptoms characteristic of the health problem or condition, may be lessened permanently, or for a short period of time. The effectiveness of a treatment of interstitial cystitis or overactive bladder can be determined using any standard index, such as those described herein, or can be determined based on the patient's subjective assessment. A patient is considered “treated” if there is a reported reduction in symptoms related to overactive bladder activity or interstitial cystitis. In one embodiment, the compounds of formula (I) and/or (II) are useful for treating interstitial cystitis or overactive bladder, as these compounds may selectively modulate the nervous system affecting sensory aspects of OAB and IC without affecting or negatively impacting motor neuron function associated with bladder and sphincter control.

The term “treatment period” means the period of time during which the drug is administered to a subject. For example, the treatment period can be from about 2 weeks to about 2 years. In some embodiments, the treatment period can be about 2, about 4, about 6, about 8, about 10, about 12, about 14, about 16, about 18, about 20, about 24, about 52, about 76 or about 104 weeks. The efficacy of the drug for OAB for example, can be assessed by measuring certain parameters and calculating the changes from baseline over the treatment period. The efficacy parameters include, but are not limited to micturitions, urge urinary incontinence episodes, total incontinence episodes, and urgency episodes.

Other diseases or symptoms that may be cured with the composition of the invention include: Underactive bladder: Also known as underactive bladder syndrome (UAB), underactive bladder includes difficulty with bladder emptying, such as hesitancy to start the stream, a poor or intermittent stream, or sensations of incomplete bladder emptying. Detrusor pressurization with strength or duration that is not sufficient for timely and efficient bladder emptying (′detrusor underactivity or ‘DU’), bladder outlet obstruction, and volume hypersensitivity (‘OAB’) are often found concurrent with UAB. Various means can be used to diagnose UAB, including a subject or patient voiding diary (to assess voided volumes and frequency of voiding) and a post-void residual volume; uninstrumented uroflow as well as a neurologic and pelvic examination; imaging for abnormal bladder morphology or vesicoureteral reflux/hydronephrosis; or invasive urodynamics. Urethra Dysfunction: Impaired urethra function, such as impairment to urine expulsion function or timing. Examples of urethra dysfunction in a subject include increased oxidative stress in the urethra, increased mitochondrial dysfunction in the urethra, or decreased urethra contractility. Urethra dysfunction can occur at any age, but is more common when the subject is an older adult, such as an adult at least 50 years old.

Urinary Frequency: The number of times of urination, such as in a day or night. “Increased urinary frequency” is eight or more times in a day, or two times or more in a night.

Urge Incontinence: Involuntary loss of urine occurring for no apparent reason while feeling urinary urgency.

Thus, in some embodiments there is provided a method of preventing/treating/amilorating urinary bladder, urinary tract or kidney disease or syndrome comprising the step of administering a hydrophilic hydrogel (phase A) into an the urinary bladder, urinary tract or kidney of a subject in need, and administering an organic solution of active agent and one or more of hydrophobic polymer, a fatty acid, a silicon, a fatty acid ester, a triglyceride, a glyceride, a phospholipid, a silicon or a wax (phase B) into the hydrophilic hydrogel (phase A) wherein a core is formed in-situ in the urinary bladder, urinary tract or kidney upon instillation of phase B into Phase A and wherein the formed solid entraps and allows an extended release of the active agent, wherein phase A or phase B or both comprises at least one active agent; thereby delivering the at least one active ingredient into the urinary bladder, urinary tract or kidney and treating the urinary bladder, urinary tract or kidney disease or syndrome. In some embodiments, the urinary bladder, urinary tract or kidney disease or syndrome include one or more of urinary tract infection, chronic cystitis, overactive bladder, partial bladder obstruction, interstitial cystitis urethritis, pain and bladder cancer or any of the other syndromes or diseases described herein. The active agent is any of the active agents suggested herein and the physician may choose the appropriate active agent according to the disease or the syndrome.

### EXAMPLES

### Example 1

TABLE 1

| | Ingredient | Parts |
|---|---|---|
| Phase A | Carbopol 980 | 0.095 |
| | 2-dimethylamino ethanol | 0.086 |
| | Deionized Water | 94.82 |
| Phase B | PLGA 50:50 ester terminated (21 dL/g) | 1.25 |
| | PLGA 50:50 ester terminated (65 dL/g) | 0.80 |
| | DMSO | 2.60 |
| | Lidocaine free base | 0.3 |
| | Oxybutynin HCl | 0.05 |
| | Total | 100 |

### Preparation of Phase A:

Carbopol was dispersed in water while mixing at 400 rpm using an overhead stirrer, to form a thin cloudy dispersion without lumps. In a separate vessel 2-dimethylamino ethanol was dissolved in part of water and the solution was added to the Carbopol dispersion, while mixing at 400 rpm using an overhead stirrer. A gel was obtained

### Preparation of Phase B

DMSO was heated to 70-80° C. in a beaker using a water bath and hot plate. PLGA was added and continuously stirred at ˜200 rpm using an overhead stirrer with a 3 blades impeller until a viscous liquid mixture was obtained. The heat was turned off and the remaining ingredients were added one by one while stirring at ˜200 rpm until dissolved. A viscous liquid mixture was obtained.

### In-Situ Drug Delivery System Formation:

Step 1: 95 g of Phase A were instilled into 100 ml vial, using a 100 ml syringe attached to a 12G catheter. Step 2: following step 1, 5 g of Phase B were instilled using a 10 ml syringe attached to a 12G catheter into phase A; a spheric solid mass containing the drugs was instantaneously generated in-situ.

### Example 2

TABLE 2

| | Ingredient | g |
|---|---|---|
| Phase A | Carbopol 974p | 0.14 |
| | TRIS Base | 0.105 |
| | Deionized Water | 69.755 |
| Phase B | PLGA 50:50 ester terminated (0.26-0.54 dL/g) | 0.7 |
| | PLGA 50:50 ester terminated (0.15-0.25 dL/g) | 1.15 |
| | POVIDON | 0.26 |
| | HPMC (Hypromellose AN5) | 0.013 |
| | Oxybutynin HCl | 0.03 |
| | Lidocaine base | 0.6 |
| | DMSO | 2.247 |

Preparation of Phase A: Carbopol was dispersed in water while mixing at 400 rpm using an overhead stirrer, to form a thin cloudy dispersion without lumps. In a separate vessel TRIS was dissolved in part of water. The solution was added to the Carbopol dispersion, while mixing at 400 rpm using an overhead stirrer. A gel was obtained with a viscosity of 2660 cPs.

### Preparation of Phase B:

DMSO was heated to 70-80° C. in a beaker using a water bath and hot plate. PLGA was added and continuously stirred at ˜200 rpm using an overhead stirrer with a 3 blades impeller until a viscous liquid mixture was obtained. The heat was turned off and the remaining ingredients were added one by one while stirring at ˜200 rpm until dissolved. A viscous liquid mixture was obtained.

### In-Situ Drug Delivery System Formation:

Step 1: 70 g of Phase A were instilled into 100 ml vial, using a 100 ml syringe attached to a 12G catheter. Step 2: following step 1, 5 g of Phase B were instilled using a 10 ml syringe attached to a 12G catheter into phase A generating in-situ a solid drug delivery system.

### Example 3

TABLE 3

| | Ingredient | g |
|---|---|---|
| Phase A | Carbopol 980 | 0.07 |
| | 2-dimethylamino ethanol | 0.063 |
| | Deionized Water | 69.867 |
| Phase B | DMSO | 2.247 |
| | PLGA 50:50 ester terminated 0.26-0.54 dL/g) | 0.7 |
| | PLGA 50:50 ester terminated 0.15-0.25 dL/g) | 1.15 |
| | POVIDON | 0.26 |
| | HPMC (Hypromellose AN5) | 0.013 |
| | Oxybutynin HCl | 0.03 |
| | Lidocaine base | 0.2 |
| | Lidocaine HCl | 0.4 |

### Preparation of Phase A:

Carbopol was dispersed in water while mixing at 400 rpm using an overhead stirrer, to form a thin cloudy dispersion without lumps. In a separate vessel 2-dimethylamino ethanol was dissolved in part of water. The solution was added to the Carbopol dispersion, while mixing at 400 rpm using an overhead stirrer. A gel was obtained.

### Preparation of Phase B:

DMSO was heated to 70-80° C. in a beaker using a water bath and hot plate. PLGA was added and continuously stirred at ˜200 rpm using an overhead stirrer with a 3 blades impeller until a viscous liquid mixture was obtained. The heat was turned off and the remaining ingredients were added one by one while stirring at ˜200 rpm until dissolved. A viscous liquid mixture was obtained.

### In-Situ Drug Delivery System Formation:

Step 1: 70 g of Phase A were instilled into 100 ml vial, using a 100 ml syringe attached to a 12G catheter.

Step 2: following step 1, 5 g of Phase B were instilled using a 10 ml syringe attached to a 12G catheter into phase A generating in-situ a spherical solid drug delivery system.

### Example 4

TABLE 4

| | Ingredient | g |
|---|---|---|
| Phase A | Carbopol 974p | 0.1 |
| | TRIS Base | 0.05 |
| | Deionized Water | 49.85 |
| Phase B | DMSO | 2.147 |
| | PLGA 50:50 ester terminated 0.26-0.54 dL/g) | 0.7 |
| | PLGA 50:50 ester terminated 0.15-0.25 dL/g) | 1.15 |
| | POVIDON | 0.26 |
| | HPMC (Hypromellose AN5) | 0.013 |
| | Sodium bicarbonate | 0.1 |
| | Oxybutynin HCl | 0.03 |
| | Lidocaine | 0.6 |

### Preparation of Phase A:

Carbopol was dispersed in water while mixing at 400 rpm using an overhead stirrer, to form a thin cloudy dispersion without lumps. In a separate vessel 2-dimethylamino ethanol was dissolved in part of water. The solution was added to the Carbopol dispersion, while mixing at 400 rpm using an overhead stirrer. A gel was obtained.

### Preparation of Phase B

Phase B was prepared at 65-75° C. throughout the procedure using a water bath and hot plate. DMSO was added into a beaker and mixed for 10 minutes using an overhead stirred with a 3 blades impeller. Each ingredient was added while mixing at ˜200 rpm until fully dissolved. The heat was turned off and allowed to cool to room temperature while mixing. A viscous liquid mixture was obtained.

### In-Situ Drug Delivery System Formation:

Step 1: 50 g of Phase A were instilled into 100 ml vial, using a 100 ml syringe attached to a 12G catheter.

Step 2: following step 1, 5 g of Phase B were instilled using a 10 ml syringe attached to a 12G catheter into phase A generating in-situ a spherical solid drug delivery system.

### Example 5

TABLE 5

| | Ingredient | w/w % |
|---|---|---|
| Phase A | Carbopol 974p | 0.25 |
| | NaOH 1N | 0.35 |
| | Deionized Water | ad 100 |
| Phase B | PLGA 50:50 ester terminated 0.26-0.54 dL/g | 14.0 |
| | PLGA 50:50 ester terminated 0.15-0.25 dL/g | 23.0 |
| | POVIDON | 4.75 |
| | HPMC (Hypromellose AN5) | 0.25 |
| | Gemcitabine | 2 |
| | DMSO | ad 100 |

### Preparation of Phase A:

Carbopol was dispersed in water while mixing at 400 rpm using an overhead stirrer to form a thin cloudy dispersion without lumps. In a separate vessel, NaOH was mixed in water. The solution was added to the Carbopol dispersion, mixing at 400 rpm using an overhead stirrer. A hydrogel was obtained.

### Preparation of Phase B:

DMSO was heated to 65-75° C. in a beaker using a water bath and hot plate. Each ingredient was added while mixing at ˜200 rpm until fully dissolved. The heat was turned off and allowed to cool to room temperature while mixing. A viscous liquid mixture was obtained.

### In-Situ Drug Delivery System Formation:

Step 1: 50 g of Phase A were instilled into 100 ml vial, using a 50 ml syringe attached to a 12G catheter.

Step 2: following step 1, 1 g of Phase B were instilled using a 2 ml syringe attached to a 12G catheter into phase A generating in-situ a solid drug delivery system.

### Example 6

TABLE 6

| | Ingredient | w/w % |
|---|---|---|
| Phase A | Carbopol 980 | 0.1 |
| | 2-dimethylamino ethanol | 0.09 |
| | Deionized Water | ad 100 |
| Phase B | DMSO | 57.4 |
| | PLGA 50:50 ester terminated 0.26-0.54 dL/g | 14.0 |
| | PLGA 50:50 ester terminated 0.15-0.25 dL/g | 23.0 |
| | POVIDON | 4.75 |
| | HPMC (Hypromellose AN5) | 0.25 |
| | Mitomycin C | 0.6 |
| | DMSO | ad 100 |

### Preparation of Phase A:

Carbopol is dispersed in water while mixing at 400 rpm using an overhead stirrer, to form a thin cloudy dispersion without lumps. In a separate vessel 2-dimethylamino ethanol is mixed in part of water. The solution is added to the Carbopol dispersion, while mixing at 400 rpm using an overhead stirrer. A hydrogel is obtained.

### Preparation of Phase B:

DMSO is heated to 70-80° C. in a beaker using a water bath and hot plate. PLGA is added and continuously stirred at ˜200 rpm using an overhead stirrer with a 3 blades impeller until a viscous liquid mixture is obtained. The heat is turned off and the remaining ingredients are added one by one while stirring at ˜200 rpm until dissolved. A viscous liquid mixture is obtained.

### In-Situ Drug Delivery System Formation:

Step 1: 50 g of Phase A is instilled into 100 ml vial, using a 50 ml syringe attached to a 12G catheter. Step 2: following step 1, 2 g of Phase B are instilled using a 5 ml syringe attached to a 12G catheter into phase A generating in-situ a solid drug delivery system.

### Example 7 In-Vitro Release of Active Agents

The in-vitro release of Oxybutynin and Lidocaine from the biphasic composition of example 2 was studied. 5 g of phase B were instilled with a 10 ml syringe attached to a 12-gauge catheter into a vial containing 70 g of Phase A. A three-dimensional (3-D) amorphous spheric structure comprising both lidocaine and Oxybutynin was formed in-situ. The vial was kept in an orbital shaker incubator at 37° C. and 20 rpm. After 3 hours, 20 mL of Artificial Urine Fluid (AUF), representative of the components of human urine in accordance with a published protocol (Sarigul et al, December 2019) were added to the vial. The vial was kept in an orbital shaker incubator at 37° C. and 20 rpm during the experiment. Samples of 85 mL were withdrawn every 24 h and replaced by fresh AUF for 8 days. Oxybutynin and lidocaine concertation in the AUF were quantitatively determined by HPLC.

### Results:

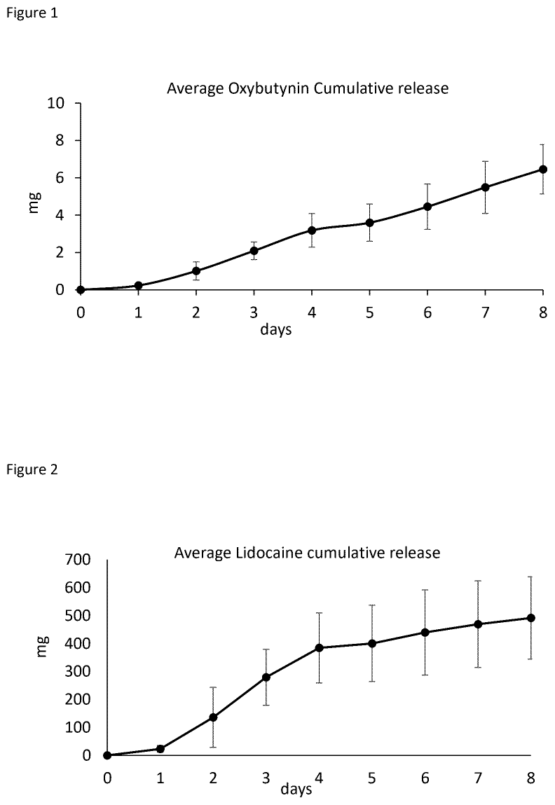

FIGS. 1 and 2 illustrate the release profile of the drug entrapped in the in-situ generated amorphous spheric mass 3-d mass structure during 8 days of experiment. As can be seen a continuous release of the two drugs is achieved in vitro using the biphasic composition of the invention.

### Example 8 In-Vivo Study

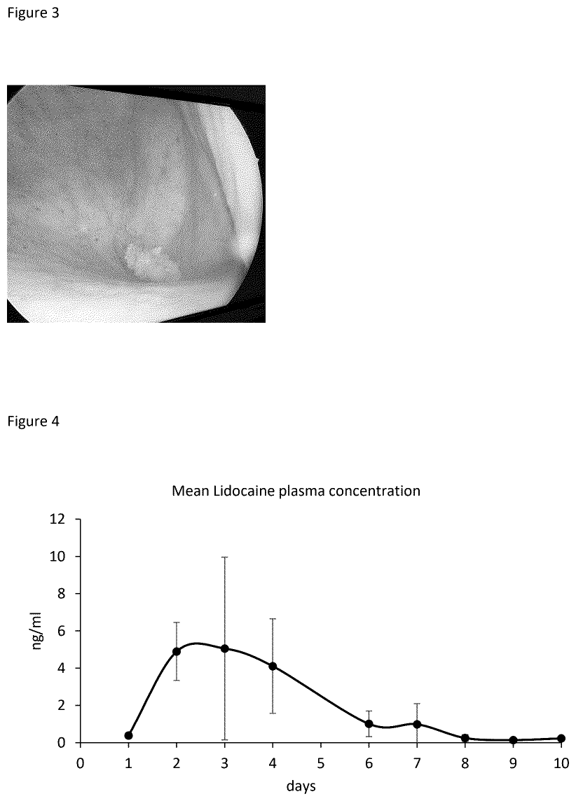

The in-vivo mean plasma concentration of Oxybutynin and Lidocaine from the biphasic composition of example 2 was studied. The plasma concentration of Oxybutynin's active metabolite, N-desethyloxybutynin, was also assessed. 50 g of phase A were instilled into urinary bladder of each of four female domestic pigs followed by instillation of 5 g phase B in each animal. Seven days post instillation, the three-dimensional (3-D) amorphous spheric mass formed in-situ was visualized using a cystoscope (FIG. 3). Blood samples were withdrawn during the 10 days experiment. Plasma concentration of Oxybutynin, N-desethyloxybutynin and Lidocaine were measured using LC-MS-MS analytical method.

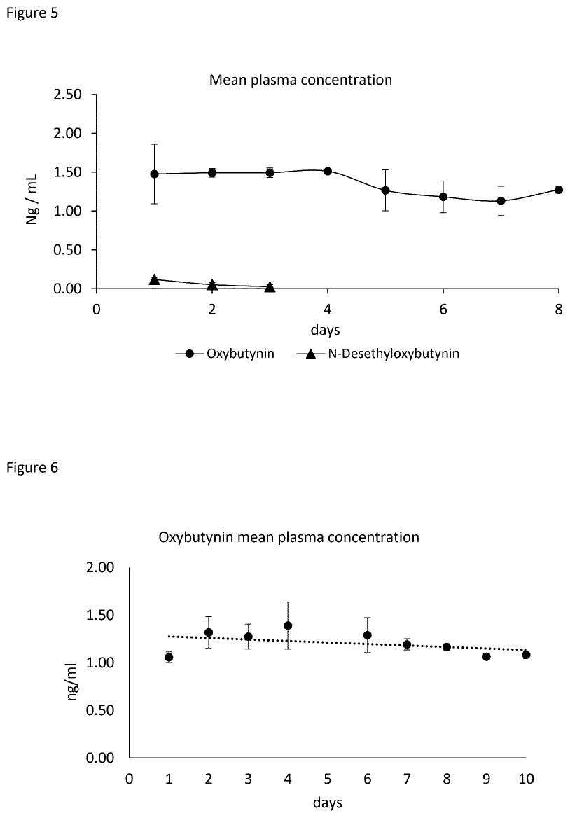

The results are presented in FIGS. 4 and 5 that illustrate the release profile of the drug entrapped in the in-situ generated 3-d mass amorphous spheric mass, during 10 days of experiment. As can be seen a continuous release of the drugs can be achieved in vivo over a period of at least 7 days using the biphasic composition of the invention. N-desethyloxybutynin concentration in the plasma was undetectable.

### Example 9

TABLE 7

| | Ingredient | % w/w |
|---|---|---|
| Phase A | Carbopol 974p | 0.1 |
| | TRIS Base | 0.09 |
| | Deionized Water | ad 100 |
| Phase B | PLGA 50:50 ester terminated (0.26-0.54 dL/g) | 14.0 |
| | PLGA 50:50 ester terminated (0.15-0.25 dL/g) | 23.0 |
| | POVIDON | 5.2 |
| | HPMC (Hypromellose AN5) | 0.5 |
| | Gemcitabine | 0.5 |
| | Cisplatin | 0.5 |
| | DMSO | ad 100 |

### Preparation of Phase A:

Carbopol is dispersed in water while mixing at 400 rpm using an overhead stirrer, to form a thin cloudy dispersion without lumps. In a separate vessel TRIS is dissolved in part of water. The solution is added to the Carbopol dispersion, while mixing at 400 rpm using an overhead stirrer. A gel is obtained.

### Preparation of Phase B:

DMSO is heated to 70-80° C. in a beaker using a silicon oil bath and hot plate. PLGA is added and continuously stirred at ˜200 rpm using an overhead stirrer with a 3 blades impeller until a viscous liquid mixture was obtained. The heat is turned off and the remaining ingredients are added one by one while stirring at ˜200 rpm until dissolved. A viscous liquid mixture is obtained.

### Example 10

TABLE 8

| | Ingredient | g |
|---|---|---|
| Phase A | Carbopol 974p | 0.1 |
| | TRIS Base | 0.05 |
| | Deionized Water | 49.85 |
| Phase B | Witepsol H15 | 1.15 |
| | Bees Wax | 1.1 |
| | Ethyl Oleate | 1.15 |
| | Span 80 | 0.10 |
| | Water | 0.25 |
| | Lidocaine | 0.25 |

### Preparation of Phase A:

Carbopol was dispersed in water while mixing at 400 rpm using an overhead stirrer, to form a thin cloudy dispersion without lumps. In a separate vessel TRIS base was dissolved in part of water. The solution was added to the Carbopol dispersion, while mixing at 400 rpm using an overhead stirrer. A gel was obtained.

### Preparation of Phase B

Phase B was prepared at 65-75° C. throughout the procedure using a water bath and hot plate. Each ingredient was added while mixing at ˜200 rpm until fully dissolved. The heat was turned off and the solution obtained was transferred into a 5 g syringe and allowed to cool to room temperature.

### In-Situ Drug Delivery System Formation:

Step 1: 50 g of Phase A were instilled into 100 ml vial, using a 50 ml syringe attached to a 12G catheter.

Step 2: following step 1, 4 g of Phase B pre-heated to 50° C. were instilled using a 5 ml syringe attached to a 12G catheter into phase A generating in-situ a solid drug delivery system.

### Example 11

TABLE 9

| | Ingredient | % w/w |
|---|---|---|
| Phase A | Carbopol 974p | 0.15 |
| | Triethanolamine | 0.15 |
| | Deionized Water | Ad 100 |
| Phase B | Eudragit L100 | 7.5 |
| | Povidon | 5.2 |
| | HPMC | 0.25 |
| | Oxybutynin HCl | 0.6 |
| | Lidocaine | 12 |
| | DMSO | ad 100 |

### Preparation of Phase A:

Carbopol was dispersed in water while mixing at 400 rpm using an overhead stirrer, to form a thin cloudy dispersion without lumps. In a separate vessel triethanolamine was mixed in part of water. The solution was added to the Carbopol dispersion, while mixing at 400 rpm using an overhead stirrer. A gel was obtained.

### Preparation of Phase B

Phase B was prepared at room temperature throughout the procedure. DMSO was placed in a beaker. Each ingredient was added while mixing at ˜600 rpm using an overhead stirrer with 3 blade impeller until fully dissolved.

### In-Situ Drug Delivery System Formation:

Step 1: 50 g of Phase A were instilled into 100 ml vial, using a 50 ml syringe attached to a 12G catheter.

Step 2: following step 1, 5.0 g of Phase B were instilled using a 10 ml syringe attached to a 12G catheter into phase A generating in-situ a solid drug delivery system.

### Example 12

TABLE 10

| | Ingredient | g |
|---|---|---|
| Phase A | Carbopol 974P | 0.6 |
| | Triethanolamine | 0.6 |
| | Deionized Water | 48.8 |
| Phase B | PEG 300 | 2.70 |
| | PLGA 50:50 ester terminated 0.26-0.54 dL/g) | 0.84 |
| | PLGA 50:50 ester terminated 0.15-0.25 dL/g) | 1.38 |
| | POVIDON | 0.312 |
| | HPMC (Hypromellose AN5) | 0.012 |
| | Oxybutynin HCl | 0.036 |
| | Lidocaine | 0.72 |

### Preparation of Phase A:

Carbopol was dispersed in water while mixing at 400 rpm using an overhead stirrer, to form a thin cloudy dispersion without lumps. In a separate vessel Triethanolamine was added in part of water. The solution was added to the Carbopol dispersion, while mixing at 400 rpm using an overhead stirrer. A gel was obtained.

### Preparation of Phase B:

PEG was heated to 70-80° C. in a beaker using a water bath and hot plate. PLGA was added and continuously stirred at ˜200 rpm using an overhead stirrer with a 3 blades impeller until a viscous liquid mixture was obtained. The heat was turned off and the remaining ingredients were added one by one while stirring at ˜200 rpm until dissolved. A viscous liquid mixture was obtained.

### In-Situ Drug Delivery System Formation:

Step 1: 50 g of Phase A were instilled into 100 ml vial, using a 100 ml syringe attached to a 12G catheter.

Step 2: following step 1, 6 g of Phase B were instilled using a 10 ml syringe attached to a 12G catheter into phase A generating in-situ a spherical solid drug delivery system.

### Example 13

TABLE 11

| | Ingredient | g |
|---|---|---|
| Phase A | Carbopol 974p | 0.1 |
| | TRIS Base | 0.05 |
| | Deionized Water | 49.85 |
| Phase B | Witepsol H15 | 1.45 |
| | Bees Wax | 1.45 |
| | Ethyl Oleate | 0.7 |
| | Lidocain | 0.25 |
| | Oxibutarine HCl | 0.05 |
| | Cremophore rh-40 | 0.1 |

### Preparation of Phase A:

Carbopol was dispersed in water while mixing at 400 rpm using an overhead stirrer, to form a thin cloudy dispersion without lumps. In a separate vessel TRIS base was dissolved in part of water. The solution was added to the Carbopol dispersion, while mixing at 400 rpm using an overhead stirrer. A gel was obtained.

### Preparation of Phase B

Phase B was prepared at 70-75° C. throughout the procedure using a water bath and hot plate. Each ingredient was added while mixing at ˜200 rpm until fully dissolved. The heat was turned off and the solution obtained was transferred into a 5 g syringe and allowed to cool to room temperature.

### In-Situ Drug Delivery System Formation:

Step 1: 50 g of Phase A were instilled into 100 ml vial, using a 50 ml syringe attached to a 12G catheter.

Step 2: following step 1, 4 g of Phase B pre-heated to 50° C. were instilled using a 5 ml syringe attached to a 12G catheter into phase A generating in-situ a solid drug delivery system.

### Example 14 In-Vivo Study

The in-vivo mean plasma concentration of Oxybutynin from the biphasic composition of example 13 was studied. 50 g of phase A were instilled into urinary bladder of each of 2 female domestic pigs followed by instillation of 5 g phase B in each animal. Blood samples were withdrawn during the 10 days experiment. Plasma concentration of Oxybutynin and it's active metabolite N-desethyloxybutynin were measured using LC-MS-MS analytical method.

The results are presented in FIG. 6 that illustrate the release profile of oxybutynin entrapped in the in-situ generated 3-d mass structure, during 10 days of experiment. As can be seen, a continuous release of oxybutynin was achieved in vivo over a period of at least 10 days using the biphasic composition of the invention while N-desethyloxybutynin plasma concentrations were very low and were undetectable after a few days.

### Example 15—Clinical Study

The clinical safety and efficacy of the below composition were studied in patients suffering from interstitial cystitis/bladder pain syndrome (IC/BPS).

TABLE 12

| Phase A | Ingredient | % w/w |
|---|---|---|
| | Carbopol 974P | 0.2 |
| | 2-dimethylamino ethanol | 0.18 |
| | Deionized Water | ad. 100 |
| Phase B | PLGA (50:50 Ester terminated 0.26-0.54 dL/g) | 14.0 |
| | PLGA (50:50 ester terminated 0.15-0.25 dL/g) | 23.0 |
| | POVIDON | 5.2 |
| | HPMC (Hypromellose AN5) | 0.25 |
| | Oxybutynin HCl | 0.6 |
| | Lidocaine Free Base | 12.0 |
| | DMSO | ad. 100 |

### Preparation of Phase

Carbopol was dispersed in part of the water while mixing at 400 rpm using an overhead stirrer, to form a thin cloudy dispersion without lumps. In a separate vessel, 2-dimethylamino ethanol was added to the remaining amount of water. The solution was added to the carbopol dispersion, while mixing at 400 rpm using an overhead stirrer. A gel with a viscosity of 2660 cPs was obtained and sterilized.

### Preparation of Phase B

DMSO was heated to 70-80° C. in a beaker using a silicon oil bath and hot plate. PLGA was added and continuously stirred at ˜200 rpm using an overhead stirrer with a 3 blades impeller until a viscous liquid mixture was obtained. The heat was turned off and the remaining ingredients were added one by one while stirring at ˜200 rpm until dissolved. A viscous liquid mixture was obtained and sterilized.

### Clinical Study

The aim of the study was to evaluate the safety and efficacy of composition described herein in Example 15 in patients with Interstitial Cystitis/Bladder Pain Syndrome (IC/BPS) in a single center single arm study. The study protocol and informed consent form were approved by the institutional review board at the participating study site. The study was conducted in accordance with the ethical principles originating in or derived from the Declaration of Helsinki and Good Clinical Practice Guidelines.

This study consisted of 4 weekly instillations (once a week). During screening, all IC/BPS patients were diagnosed according to the American Urological Association (AUA) Guideline. All IC/BPS patients reported symptoms of at least 6 weeks duration prior to treatment. All patients had previously failed with other therapies and suffered from symptoms for many years.

During treatment period, before administration of the composition, residual urine in the bladder was removed using a urethral catheter. Subsequently, 50 ml of phase A were instilled while securely holding the catheter funnel connected to the syringe containing Phase A. Once instilled, the syringe was disconnected from the catheter funnel. Right after, 5 ml of Phase B were instilled while securely holding the catheter funnel connected to the syringe containing Phase B. Once instilled, the syringe was disconnected from the catheter funnel and the catheter was removed.

For efficacy assessment, patients were asked to complete a Numeric Rating Scale (NRS) for bladder pain scoring and the O'Leary-Sant Interstitial Cystitis Symptom Index (ICSI) for their quality-of-life assessment. Urinary urgency was measured by voiding frequency over 24 hours recorded in voiding log. The efficacy endpoints were reduction at EOS from baseline in NRS score for pain, ICSI score and urinary urgency.

### Patients

A total of 10 patients were enrolled to receive the composition of example 15.

### Results

### Efficacy

Patients tolerated the treatments very well and compliance meet expectations. The reduction in pain median as measured by NRS Score, was from 7.5 (Mean 7, SD 1.63) before treatment to 3. (Mean 3.4, SD 2.27), at EOS (p=0.005). The change in the median ICSI score was from 27.5 (Mean 27.5, SD 4.48) at baseline to 22 (Mean 18.9, SD 6.19) at EOS (p=0.012). The change in the voiding frequency median score was from 17.5 (Mean 15.9, SD 4.36) at baseline to 10.5 (Mean 10.7, SD 4.11) at EOS (p=0.008).

TABLE 13

| | | | |
|---|---|---|---|
| | | | Standard |
| N | Mean | Median | deviation |

NRS SCORE

| | | | | |
|---|---|---|---|---|
| Baseline | 10 | 7 | 7.5 | 1.63 |
| EOS | 10 | 3.4 | 3 | 2.27 |

ICSI SCORE

| | | | | |
|---|---|---|---|---|
| Baseline | 10 | 27.5 | 27.5 | 4.48 |
| EOS | 10 | 18.9 | 22 | 6.19 |

Voiding frequency

| | | | | |
|---|---|---|---|---|
| Baseline | 10 | 15.9 | 17.5 | 4.36 |
| EOS | 10 | 10.7 | 10.5 | 4.11 |

### Example 16—Clinical Study

The clinical safety and efficacy of the composition of example 15 were studied in patients suffering from pain associated with ureteral stenting following Endourology Procedures (EUP).

The aim of the study was to evaluate the safety and efficacy of composition from example 15 in patients suffering from pain associated with ureteral stenting following endourology procedures (EUP) in a single center single arm study. The study protocol and informed consent form were approved by the institutional review board at the participating study site. The study was conducted in accordance with the ethical principles originating in or derived from the Declaration of Helsinki and Good Clinical Practice Guidelines.

Patients suffering from pain associated with ureteral stenting following EUP were treated once. An end of study (EOS) visit was carried out seven days post-treatment.

Patients received a single treatment. Before administration of the study drug, residual urine in the bladder was removed using a urethral catheter. Subsequently, 50 ml of phase A were instilled while securely holding the catheter funnel connected to the syringe containing Phase A. Once instilled, the syringe was disconnected from the catheter funnel. Right after, 5 ml of Phase B were instilled while securely holding the catheter funnel connected to the syringe containing Phase B. Once instilled, the syringe was disconnected from the catheter funnel and the catheter was removed.

### Efficacy Assessment

For efficacy assessment, patients were asked to complete a Numeric Rating Scale (NRS) for bladder pain scoring.

Data results were assessed for statistical significance using Wilcoxon two tailed, paired nonparametric statistical test between the baseline and ESO groups. Statistical assessment was considered significant at P<0.05. Statistical analyses were performed using DATAtab (DATAtab Team (2021). DATAtab e.U. Graz, Austria. URL https://datatab.net/).

### Results

Patients—a total of 16 patients ages>18 years old were enrolled to receive the treatment.

### Efficacy Assessment

Patients tolerated the treatment very well and met compliance expectations. The reduction in pain median as measured by NRS Score, was from 9 (Mean 8.56, SD 1.67) before treatment to 5 (Mean 5.06, SD 2.52), at EOS. The statistical significance was p=0.001.

TABLE 14

| | | | | Standard |
|---|---|---|---|---|
| | N | Mean | Median | deviation |
| Baseline | 16 | 8.56 | 9 | 1.67 |
| EOS | 16 | 5.06 | 5 | 2.52 |

### Example 17 Clinical Study

The clinical safety and efficacy of the composition of example 15 were studied in patients suffering from over active bladder (OAB).

The aim of the study was to evaluate the safety and efficacy of the composition from example 15 in patients suffering from Over Active Bladder (OAB) in a single center single arm study. The study protocol and informed consent form were approved by the institutional review board at the participating study site. The study was conducted in accordance with the ethical principles originating in or derived from the Declaration of Helsinki and Good Clinical Practice Guidelines.

Patients suffering from over active bladder (OAB) received 4 weekly instillations (once a week) treatment. Before administration of the study drug, residual urine in the bladder was removed using a urethral catheter. Subsequently, 50 ml of phase A were instilled while securely holding the catheter funnel connected to the syringe containing Phase A. Once instilled, the syringe was disconnected from the catheter funnel. Right after, 5 ml of Phase B were instilled while securely holding the catheter funnel connected to the syringe containing Phase B. Once instilled, the syringe was disconnected from the catheter funnel and the catheter was removed.

### Efficacy Assessment

For efficacy assessment, urinary urgency was measured by voiding frequency over 24 hours recorded in voiding log.

Patients: 4 patients ages>18 years old were enrolled to receive the treatment.

### Results

### Efficacy Assessment

Patients tolerated the treatment very well and met compliance expectations. The change in the voiding frequency was marked in all 4 patients (Table 15). One patient reported his nocturnal enuresis has stopped. Another patient reported the stopping of his incontinence.

TABLE 15

| | |
|---|---|
| Patient | Number of voiding episodes/24 h |

| No. | Baseline | EOS |
|---|---|---|
| 1 | 24 | 12 |
| 2 | 8 | 5 |
| 3 | 8 | 4 |
| 4 | 18 | 10 |

### Example 18

TABLE 16

| | Ingredient | % w/w |
|---|---|---|
| Phase A | Carbopol 974P | 0.2 |
| | 2-dimethylamino ethanol | 0.18 |
| | Deionized Water | ad. 100 |
| Phase B | PLGA (50:50 Ester terminated 0.26-0.54 dL/g) | 14.0 |
| | PLGA (50:50 ester terminated 0.15-0.25 dL/g) | 23.0 |
| | POVIDON | 5.2 |
| | HPMC (Hypromellose AN5) | 0.25 |
| | Docetaxel | 0.5 |
| | Gemcitabine | 0.5 |
| | DMSO | ad. 100 |

Preparation of the composition was done is a similar manner to the preparation described in Example 1.

### Cytotoxicity (MTT Assay)

The cytotoxicity on T24 cell line in different release time intervals was studied using medium samples taken from the in-vitro release study of composition example No 18.

1.0 g of Phase B was instilled with a two ml syringe connected to a 12-gauge catheter into a vial containing 5.0 g of Phase A. A bulky solid three-dimensional structure was formed in situ. Ten minutes after creating the solid, 30 ml of PBS medium was added to the vial. The vial was kept in a rotating shaker at 37° C. and 10 rpm. At 3, 24, 48, and 72 hours, the medium was withdrawn sampled and replaced with fresh PBS.

Cell culture. Cells of human urinary bladder cancer T24 cell line1 were cultured in the appropriate cell culture medium supplemented with 10% fetal calf serum, 2 mM L-glutamine, 100 units/mL penicillin and 100 μg/mL streptomycin (SKOV-3-luc in McCoy's in MEM+0.01 mM NEAA). Cells were kept at 37° C. in 5% CO2 and 95% humidity.

Cytotoxicity (MTT assay). Approximately 5,000 cells/well were seeded in a 96-well plate. The cells were allowed to attach overnight, a fresh medium containing appropriate dilutions of test compounds was added.

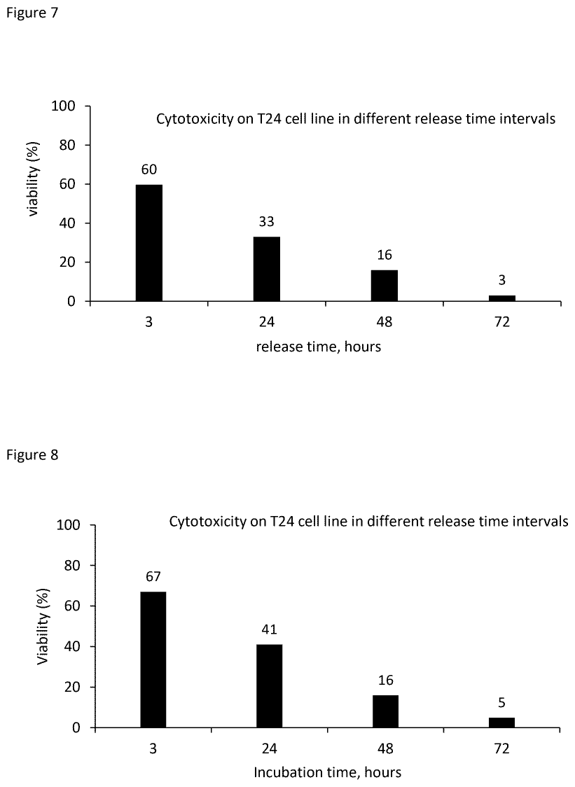

The cytotoxic activity of the released drugs from composition example 18 in time intervals 3, 24, 48, and 72 hours were tested using the MTT assay. Samples from the released medium were incubated for 24 hours. Next, the medium was discarded, and cells were further treated for one hour with 0.5 mg/ml MTT in phosphate buffer solution (PBS). Finally, the developed dye was dissolved in DMSO, and absorbance was measured at 570 nm by a microplate reader. The results shown in FIG. 7 demonstrate the cytotoxic effect of the sustained release of drugs from composition example 18.

### Example 19

TABLE 17

| | Ingredient | % w/w |
|---|---|---|
| Phase A | Carbopol 974P | 0.2 |
| | 2-dimethylamino ethanol | 0.18 |
| | Deionized Water | ad. 100 |
| Phase B | PLGA (50:50 Ester terminated 0.26-0.54 dL/g) | 14.0 |
| | PLGA (50:50 ester terminated 0.15-0.25 dL/g) | 23.0 |
| | POVIDON | 5.2 |
| | HPMC (Hypromellose AN5) | 0.25 |
| | Docetaxel | 0.1 |
| | Gemcitabine | 0.1 |
| | DMSO | ad. 100 |

Preparation of the composition was done is a similar manner to the preparation escribed in Example 1.

The cytotoxicity of composition of example 19 on T24 cell line in different release time intervals was studied using medium samples taken from the in-vitro release study in a similar manner as described in in example 18.

The results shown in FIG. 8 demonstrate cytotoxic effect of the sustained release of drugs from composition of example 19.

### Example 20

TABLE 18

| | Ingredient | % w/w |
|---|---|---|
| Phase A | Carbopol 974P | 0.2 |
| | 2-dimethylamino ethanol | 0.18 |
| | Deionized Water | ad. 100 |
| Phase B | PLGA (50:50 Ester terminated 0.26-0.54 dL/g) | 14.0 |
| | PLGA (50:50 ester terminated 0.15-0.25 dL/g) | 23.0 |
| | POVIDON | 5.2 |
| | HPMC (Hypromellose AN5) | 0.25 |
| | Docetaxel | 0.5 |
| | Gemcitabine | 0.5 |
| | Cisplatin | 0.5 |
| | DMSO | ad. 100 |

Preparation of the composition was done is a similar manner to the preparation described in Example 1.

The cytotoxicity of composition of example 20 on T24 cell line in different release time intervals was studied using medium samples taken from the in-vitro release study in a similar manner as described in in example 18.

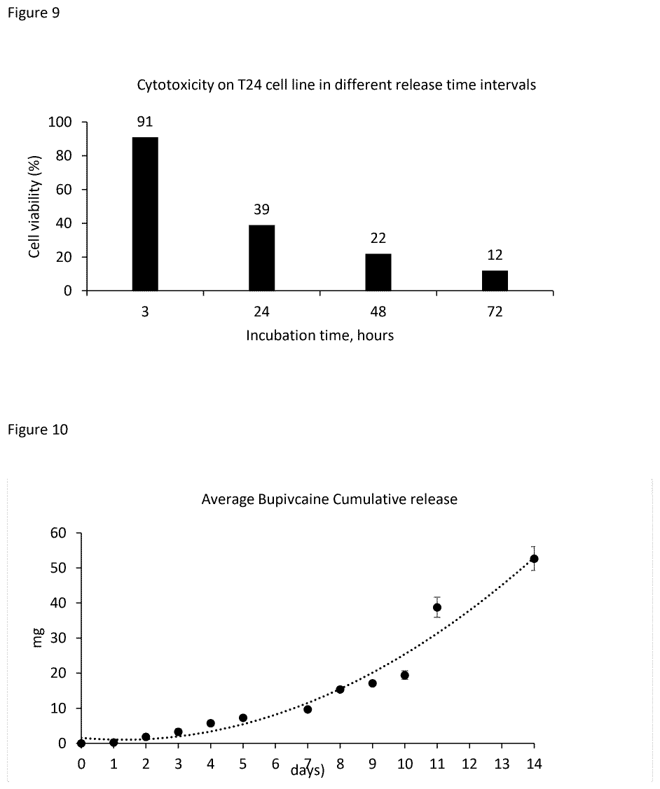

The results shown in FIG. 9 demonstrate the cytotoxic effect of the sustained release of drugs from composition example 20.

### Example 21

TABLE 19

| | Ingredient | % w/w |
|---|---|---|
| Phase A | Sodium alginate | 0.8 |
| | Calcium Carbonate | 0.7 |
| | Deionized Water | ad. 100 |
| Phase B | PLGA (50:50 Ester terminated 0.26-0.54 dL/g) | 14.0 |
| | PLGA (50:50 ester terminated 0.15-0.25 dL/g) | 23.0 |
| | POVIDON | 2.0 |
| | Solifenacin | 0.5 |
| | DMSO | ad. 100 |

### Preparation of Phase A

Sodium Alginate is dispersed in about half the amount of the water while mixing at 100 rpm using an overhead stirrer. In a separate vessel calcium carbonate is dissolved in the rest of the water. The solution is added to the alginate part, while mixing at 100 rpm using an overhead stirrer. A gel is obtained.

### Preparation of Phase B

DMSO is heated to 70-80° C. in a beaker using a silicon oil bath and hot plate. PLGA is added and continuously stirred at ˜200 rpm using an overhead stirrer with a 3 blades impeller until a viscous liquid mixture is obtained. The heat is turned off and the remaining ingredients are added one by one while stirring at ˜200 rpm until dissolved. A viscous liquid mixture is obtained and sterilized.

### Example 22

TABLE 20

| | Ingredient | % w/w |
|---|---|---|
| Phase A | Carbopol 974P | 0.2 |
| | 2-dimethylamino ethanol | 0.18 |
| | Deionized Water | ad. 100 |
| Phase B | PLGA (75:25 acid terminated 0.3-0.44 dL/g) | 14.0 |
| | PLGA (50:50 acid terminated 0.16-0.24 dL/g) | 23.0 |
| | POVIDON | 5.2 |
| | Sodium Diclofenac | 3 |
| | DMSO | ad. 100 |

Preparation of Phase A and Phase B is done is a similar manner to example 1.

### Example 23

TABLE 21

| | Ingredient | % w/w |
|---|---|---|
| Phase A | Carbopol 974P | 0.2 |
| | 2-dimethylamino ethanol | 0.18 |
| | Deionized Water | ad. 100 |
| Phase B | PLGA (75:25 acid terminated 0.9-1.3 dL/g) | 7.0 |
| | PLGA (75:25 acid terminated 0.16-0.24 dL/g) | 18.0 |
| | Fesoterodine | 0.4 |
| | DMSO | ad. 100 |

Preparation of Phase A and Phase B is done is a similar manner to example 1.

### Example 24 In-Vitro Release

TABLE 22

| | Ingredient | % w/w |
|---|---|---|
| Phase A | Carbopol 974P | 0.2 |
| | 2-dimethylamino ethanol | 0.18 |
| | Deionized Water | ad. 100 |
| Phase B | PLGA (50:50 Ester terminated 0.26-0.54 dL/g) | 14.0 |
| | PLGA (50:50 ester terminated 0.15-0.25 dL/g) | 23.0 |
| | POVIDON | 5.2 |
| | HPMC (Hypromellose AN5) | 0.25 |
| | Oxybutynin HCl | 0.6 |
| | Bupivacaine | 3.0 |
| | DMSO | ad. 100 |

Preparation of the composition was done is a similar manner to the preparation described in example 1.

The in-vitro release of bupivacaine from the composition of example 24 was studied. 5 g of phase B were instilled with a 10 ml syringe attached to a 12-gauge catheter into a vial containing 70 g of Phase A. A three-dimensional (3-D) amorphous spheric mass was formed in-situ. The vial was kept in an orbital shaker incubator at 37° C. and 20 rpm. After 3 hours, 20 ml of Artificial Urine Fluid (AUF), were added to the vial. The vial was kept in an orbital shaker incubator at 37° C. and 20 rpm during the experiment. Samples of 85 mL were withdrawn every 24 h and replaced by fresh AUF for 14 days. Bupivacaine concertation in the AUF were quantitatively determined by HPLC.

### Results:

FIG. 10 illustrates the release profile of the drug entrapped in the in-situ generated 3-d mass amorphous spheric mass during 14 days of experiment. As can be seen, a continuous release of the drug is achieved in vitro using the composition described herein.

### Example 25

TABLE 23

| | Ingredient | % w/w |
|---|---|---|
| Phase A | Carbopol 974p | 0.3 |
| | TRIS Base | 0.4 |
| | Deionized Water | Ad 100 |
| Phase B | PLGA 50:50 ester terminated 0.26-0.54 dL/g) | 23 |
| | PLGA 50:50 ester terminated 0.15-0.25 dL/g) | 14 |
| | Povidon | 5.2 |
| | HPMC | 0.25 |
| | Oxybutynin HCL | 0.6 |
| | Lidocaine | 12 |
| | DMSO | ad 100 |

Preparation of Phase A and Phase B was done in a similar manner to example 2.

### Example 26

TABLE 24

| | Ingredient | % w/w |
|---|---|---|
| Phase A | HPMC | 2.0 |
| | Deionized Water | ad. 100 |
| Phase B | PLGA (65:35 Ester terminated 0.83-0.93 dL/g) | 8.0 |
| | PLGA (65:35 ester terminated 0.55-0.75 dL/g) | 20.0 |
| | Triamcinolone Acetonide | 0.1 |
| | N-methyl-2-pyrrolidone (NMP) | ad. 100 |

### Preparation of Phase A:

The aqueous phase is heated to 80-90° C. HPMC is gradually added to the heated water with vigorous stirring. The hot dispersion aqueous solution is removed to a cold stirrer and mixed at 400 rpm to form a transparent solution without lumps. A hydrogel is obtained.

### Preparation of Phase B:

NMP is heated to 70-80° C. in a beaker using a water bath and hot plate. PLGA is added and continuously stirred at ˜200 rpm using an overhead stirrer with a three blades impeller until a viscous liquid mixture is obtained. The heat is turned off, and the remaining ingredients are added one by one while stirring at ˜200 rpm until dissolved. A viscous liquid mixture is obtained.

### Example 27

TABLE 25

| | Ingredient | g |
|---|---|---|
| Phase A | Carbopol 940 | 0.6 |
| | Xanthan Gum | 0.1 |
| | Lidocaine free base | 0.5 |
| | Deionized Water | 48.8 |
| Phase B | PEG 400 | 2.70 |
| | PLGA 75:25 ester terminated 0.8-1.2 dL/g) | 1.54 |
| | Oxybutynin HCl | 0.04 |
| | Lidocaine HCl | 0.72 |

### Preparation of Phase A:

Carbopol and xanthan gum are dispersed in about two thirds of the water while mixing at 200 rpm using an overhead stirrer, to form a thin cloudy dispersion without lumps. In a separate vessel lidocaine base is added to the remaining water. The lidocaine solution is added to the carbopol and xanthan gum dispersion, while mixing at 300 rpm using an overhead stirrer. A gel is obtained.

### Preparation of Phase B:

PEG 400 is heated to 70-80° C. in a beaker using a silicon oil bath and hot plate. PLGA is added and continuously stirred at ˜200 rpm using an overhead stirrer with a 3 blades impeller until a viscous liquid mixture is obtained. The heat is turned off and the remaining ingredients are added one by one while stirring at ˜200 rpm until they are dissolved. A viscous liquid mixture is obtained.

### Example 28

TABLE 26

| | Ingredient | % w/w |
|---|---|---|
| Phase A | Carbopol 940 | 0.3 |
| | TRIS Base | 0.4 |
| | Deionized Water | Ad 100 |
| Phase B | PLGA (50:50 Ester terminated 0.26-0.54 dL/g) | 10 |
| | PLGA (50:50 ester terminated 0.15-0.25 dL/g) | 24 |
| | Pembrolizumab | 0.4 |
| | DMSO | Ad 100 |

### Preparation of Phase A:

Carbopol is dispersed in about two thirds of the water while mixing at 200 rpm using an overhead stirrer, to form a thin cloudy dispersion without lumps. In a separate vessel TRIS base is added to the remaining water. The TRIS solution is added to the carbopol dispersion, while mixing at 300 rpm using an overhead stirrer. A gel is obtained.

### Preparation of Phase B:

70% of the amount of DMSO is heated to 70-80° C. in a beaker using a silicon oil bath and hot plate. PLGA is added and continuously stirred at ˜200 rpm using an overhead stirrer with a 3 blades impeller until a viscous liquid mixture is obtained. The heat is turned off and mixture is allowed to cool to room temperature.

About 60 minutes before intended instillation, Pembrolizumab is mixed with the remaining 30% of DMSO. The mixture is combined and mixed with the PLGA/DMSO solution.

Upon treatment, 70 g of phase A are instilled into the bladder and right after, 5 g of Phase B are instilled into Phase A. An in-situ amorphous spheric mass is formed within the bladder.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. It is to be understood that further trials are being conducted to establish clinical effects.

1.-29. (canceled)

30. A biphasic composition for instillation into the urinary bladder, or kidney for delivering an active agent comprising:
Phase A, wherein phase A comprises a hydrophilic hydrogel; and
Phase B, wherein phase B comprises an organic solution, an active agent and one or more of:
a hydrophobic polymer;
a lipid; or
a silicon;
wherein a core is formed in-situ in the urinary bladder or kidney upon instillation of phase B into Phase A and wherein the formed core entraps and allows an extended release of the active agent.

31. The biphasic composition of claim 30, wherein Phase A comprising one or more hydrophilic gelling agent selected from the group consisting of carbomer, polyacryl acid, acrylate polymers, carrageenan, polyvinyl alcohol, polyethylene glycol, sodium polyacylatemethylcellulose, hydroxypropyl methylcellulose, chitosan, guar gum, xanthan gum, gelatin, water, and optionally an alkali neutralizer.

32. The biphasic composition of claim 30, wherein the hydrophobic polymer in phase B is polyglycolic acid, polylactic acid, copolymer of polylactic acid (PLA) and polyglycolic acid (PGA), Poly(DL-lactide) poly(lactide-co-glycolide), Poly(L-lactide), Poly(ε-caprolactone) Poly(DL-lactide-co-ε-caprolactone), methacrylic acid-methyl methacrylate copolymer and any combination thereof.

33. The biphasic composition of claim 30, wherein the organic solution is dimethyl sulfoxide (DMSO), N-methyl-2-pyrrolidone, ethyl acetate, polyethylene glycol, alcohol, propylene glycol, ethyl oleate, oleic acid, liquid hydrocarbon and any combination thereof.

34. The biphasic composition of claim 30, wherein the lipid is a wax, bees wax, Witepsol™, lauric acid, cethyl palmitate, a fatty acid, a fatty acid ester, a triglyceride, a glyceride, a phospholipid or any combination.

35. The biphasic composition of claim 30, wherein the liquid organic solution of phase B is DMSO.

36. The biphasic composition of claim 30, wherein phase B comprising at least two PLGA co-polymers.

37. The biphasic composition of claim 36, wherein the PLGA co-polymers have a monomer ratio compositions from 50:50 up to 85:15 poly(lactide-co-glycolide).

38. The biphasic composition of claim 37, wherein the PLGA co-polymers have an inherent viscosity range from 0.15 to 0.95 dL/g.

39. The biphasic composition of claim 37, wherein the PLGA co-polymers have acid or hydroxy or ester end groups.

40. The biphasic composition of claim 37, comprising 0.1-45% w/w PLGA with inherent viscosity range from 0.15-0.25 dL/g, 0.1-35% w/w; and PLGA with inherent viscosity range from 0.26 to 0.54 dL/g.

41. The biphasic composition of claim 37, comprising 0.1-45% w/w PLGA with inherent viscosity range from 0.15-0.25 dL/g, 0.1-35% w/w; and PLGA with inherent viscosity range from 0.55 to 0.75 dL/g.

42. The biphasic composition of claim 30, wherein the active ingredient is anesthetic agent, analgesic agent, antimuscarinic agent, beta-2 agonist agent, an anti-cancer agent or any combination thereof.

43. The biphasic composition of claim 30, wherein said active agent is chosen from the group consisting of Streptococcus pyogenes, mitomycin C, deoxrubicin, valrubicin, cisplatin, gemcitabine, thiotepa, ethoglucid (Epodyl), epirubicin, pirarubicin, apaziquone, docetaxel and vicinium and any combination thereof.

44. The biphasic composition of claim 30, wherein the active agent is released continuously for 1, 2, 3, 4, 5, 6, 7, 8, 9, 10, 14, 21, 30 or more days.

45. The biphasic composition of claim 31, wherein the alkali neutralizer is selected from sodium hydroxide, triethanolamine, diisoprpanoamine, ammonium hydroxide, 2-dimethylamino ethanol, TRIS base, monoisopropanolamine or borax.

46. A method of forming a biphasic composition according to claim 30 comprising:
dispersing hydrophilic gelling agent in water or any other suitable hydrophilic solvent (phase A);
mixing liquid organic solution of hydrophobic polymer, a fatty acid, a fatty acid ester, a silicon. a triglyceride, a glyceride, a phospholipid, a silicon, or a wax until a viscous liquid mixture is obtained (phase B); and
injecting phase B into phase A, wherein phase A and/or phase B comprises an active agent.

47. A kit comprising: a hydrophilic hydrogel; an organic solvent and one or more of hydrophobic polymer, a fatty acid, a fatty acid ester, a silicon, a triglyceride, a glyceride, a phospholipid or a wax; and a leaflet explaining the preparation of in-situ biphasic composition.

48. The kit of claim 47, further comprising an active agent.

49. A method of preventing/treating/amilorating urinary bladder, urinary tract or kidney disease or syndrome comprising the step of administering a hydrophilic hydrogel (phase A) into an the urinary bladder, urinary tract or kidney of a subject in need, and one or more of hydrophobic polymer, a fatty acid, a silicon, a fatty acid ester, a triglyceride, a glyceride, a phospholipid, a silicon or a wax (phase B) into the hydrophilic hydrogel (phase A) wherein a core is formed in-situ in the urinary bladder, urinary tract or kidney upon instillation of phase B into Phase A and wherein the formed solid entraps and allows an extended release of the active agent, wherein phase A or phase B or both comprises at least one active agent; thereby delivering the at least one active ingredient into the urinary bladder, urinary tract or kidney and treating the urinary bladder, urinary tract or kidney disease or syndrome.

50. The method of claim 49, wherein the urinary bladder, urinary tract or kidney disease or syndrome include one or more of urinary tract infection, chronic cystitis, overactive bladder, partial bladder obstruction, interstitial cystitis urethritis, pain and bladder cancer.

51. The method of claim 49, wherein the active ingredient is gemcitabine, docetaxel or a combination thereof.
